(19) United States
(12) Patent Application Publication
(10) Pub. No.: US 2025/0042892 A1
(43) Pub. Date: Feb. 6, 2025

(54) PREPARATION OF INDOLONE-3YL-HYDRAZONO-SUBSTITUTED 1,3-THIAZOLIDINONE DERIVATIVES AND APPLICATION THEREOF

(71) Applicant: Fudan University, Shanghai (CN)

(72) Inventors: Ge MENG, Shanghai (CN); Weijuan GE, Shanghai (CN); Jing TONG, Shanghai (CN); Yanan CHENG, Shanghai (CN); Huiling CAO, Shanghai (CN)

(21) Appl. No.: 18/807,978

(22) Filed: Aug. 18, 2024

Related U.S. Application Data
(63) Continuation of application No. PCT/CN2023/078485, filed on Feb. 27, 2023.

(30) Foreign Application Priority Data
Feb. 27, 2022 (CN) 202210181997.X

(51) Int. Cl. C07D 417/12; A61K 31/427

(52) U.S. Cl. CPC C07D 417/12; A61K 31/427

(57) ABSTRACT

A series of 2-(5-substituted-indolone-3-yl-hydrozono)-3-substituted-arylidene-1,3-thiazolidinone derivatives represented by

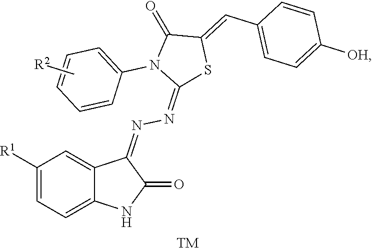

where R¹ is Cl, F, Br or CH₃, and R² is 4-F, 3-Cl, 3-CF₃ or 4-C(CH₃)₃. A pharmaceutical composition including such 2-(5-substituted-indolone-3-yl-hydrozono)-3-substituted-arylidene-1,3-thiazolidinone derivative, or a pharmaceutically-acceptable salt thereof is provided. A preparation method of such derivative and an application of such derivative as a lead compound in the preparation of PARP14 inhibitors are further provided.

### CROSS-REFERENCE TO RELATED APPLICATIONS

This application is a continuation of International Patent Application No. PCT/CN2023/078485, filed on Feb. 27, 2023, which claims the benefit of priority from Chinese Patent Application No. 202210181997.X, filed on Feb. 27, 2022. The content of the aforementioned application, including any intervening amendments thereto, is incorporated herein by reference in its entirety.

### TECHNICAL FIELD

This application relates to pharmaceuticals, and more particularly to an indolone-substituted 1,3-thiazolidinone derivative, and a preparation and application thereof.

### BACKGROUND

ADP ribosylation (ADPr) is a reversible and evolutionarily conserved post-transcriptional modification process of proteins, which is involved in the regulation of a variety of biological processes in vivo, and plays an important role in maintaining the gene stability and regulating the apoptosis. The ADPr is mainly catalyzed by members of the ADP-ribosyltransferase (ART) superfamily, where the Poly ADP-ribose polymerase (PARP) family has attracted the greatest interest. The PARP family is widely found in eukaryotic cells, and contains a highly-conserved catalytic domain (Gibson, B. A.; Kraus, W. L., New insights into the molecular and cellular functions of poly(ADP-ribose) and PARPs. Nat. Rev. Mol. Cell Biol. 2012, 13 (7), 411-424.). Based on the structural homology to the catalytic domain of PARP1, which is the initial member of the family, there are 18 members found in mammals, and their proteins are respectively named as PARP 1-18 subtypes. The PARP family is extremely important in cells, and plays an important role in maintaining genetic stability and telomere length and triggering the cellular response to external stimuli.

PARP family members can be categorized into the following four main types based on their structural domains and functional characteristics:
1) DNA-dependent PARPs: PARP1 (Lord, C. J.; Ashworth, A., PARP inhibitors: Synthetic lethality in the clinic. Science (Washington, DC, U. S.) 2017, 355 (6330), 1152-1158.), PARP2 (Chen, Q.; Kassab, M. A.; Yu, X.; Dantzer, F., PARP2 mediates branched poly ADP-ribosylation in response to DNA damage. Nat Commun 2018, 9 (1), 3233.) and PARP3;
2) Tankyrase: PARP5a (Tankyrase1) and PARP5b (Tankyrase2);
3) Cys-Cys-Cys-His (CCCH) PARPs: PARP7, PARP12, and PARP13; and
4) macro PARPs: PARP9, PARP14 (Iwata, H.; Goettsch, C.; Sharma, A.; Ricchiuto, P.; Goh, W. W. B.; Halu, A.; Yamada, I.; Yoshida, H.; Hara, T.; Wei, M.; Inoue, N.; Fukuda, D.; Mojcher, A.; Mattson, P. C.; Barabasi, A.-L.; Boothby, M.; Aikawa, E.; Singh, S. A.; Aikawa, M., PARP9 and PARP14 cross-regulate macrophage activation via STAT1 ADP-ribosylation. Nat. Commun. 2016, 7, 12849.), and PARP15.

Not all members of the PARP family have the ADP ribosyltransferase activity, and some members (e.g., PARP14) act as a mono-ADP ribosyltransferase rather than a poly-ADP ribosyltransferase.

Therefore, extensive attempts have been made to the design and synthesis of small molecule PARP inhibitors with high subtype selectivity based on structural and functional differences among members of the PARP family (Wang, S-R.; Xue, X-W., PARP Family and Clinically Used PARP Inhibitors. Guangdong Chemical Industry. 2019, 46 (9), 3). The currently-available PARP inhibitors are mainly designed for PAPR1, and the representative drugs, based on basic structural features, include benzimidazole carboxamides (1), phthalazinones (2), tricyclic indole lactams (3), indazole carboxamides (4), carbazolo diimides (5), and nicotinamide analogs (6), whose targets are mainly DNA-dependent domains and other different structural domains (FIG. 2). Although these inhibitors have different inhibitory effects on different subtypes, and some inhibitors exhibit a certain subtype-selectivity, they are still ineffective against the PARP14, which has a mono-ADP-ribosyltransferase (MARP) function (Lord, C. J.; Ashworth, A., PARP inhibitors: Synthetic lethality in the clinic. Science (Washington, DC, U. S.) 2017, 355 (6330), 1152-1158.). The commercially-available clinically-approved PARP1 inhibitors are structurally shown as follows:

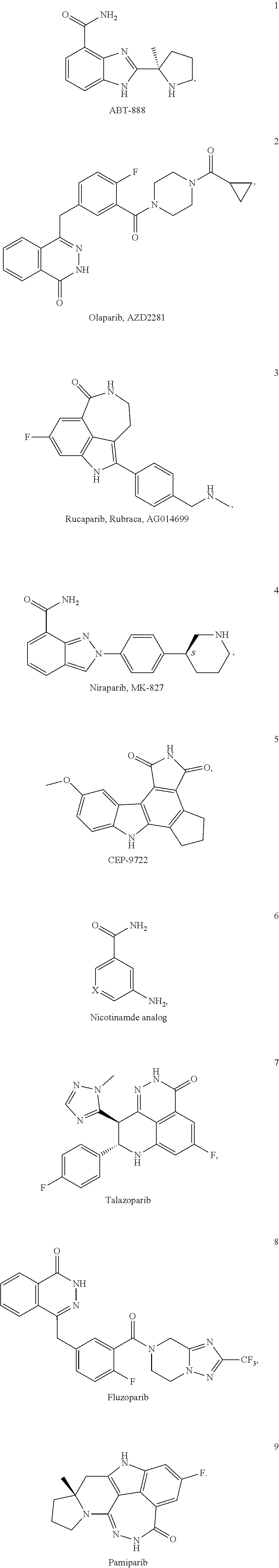

PARP14 is a DNA damage repair factor that plays a critical role in the tumor cell damage repair. Based on the critical role of PARP14 in the process of tumor cell proliferation and interaction with other inflammatory factors, the PARP14-targeting inhibitors have emerged as a new strategy in the development of antitumor drugs and therapeutic drugs for allergic inflammation (Qin, W.; Wu, H.-J.; Cao, L.-Q.; Li, H.-J.; He, C.-X.; Zhao, D.; Xing, L.; Li, P.-Q.; Jin, X.; Cao, H.-L., Research progress on PARP14 as a drug target. Front. Pharmacol. 2019, 10, 172.). Structurally, the PARP14 consists of five domains, where the catalytic domain is responsible for catalyzing the mono-ADP-ribosylation modification, and is considered as a novel target for the design of antitumor drugs (Feijs, K. L. H.; Forst, A. H.; Verheugd, P.; Luescher, B., Macrodomain-containing proteins: regulating new intracellular functions of mono(ADP-ribosyl)ation. Nat. Rev. Mol. Cell Biol. 2013, 14 (7), 445-453.). These binding domains which are closely related to PARP1 inhibitors, as suggested by structural bioinformatics analysis, are shown in FIG. 1 (Gibson, B. A.; Kraus, W. L., New insights into the molecular and cellular functions of poly(ADP-ribose) and PARPs. Nat. Rev. Mol. Cell Biol. 2012, 13 (7), 411-424.).

Almost all of the available clinical drugs target the PAPR1. The inhibitors targeting at PARP14 selectively has been rarely reported. Therefore, the search for novel PARP14 inhibitor with the new structure, the excellent site-specificity, the high potency and the low toxicity has become a research hotspot. Both H8 and H10 are selective PARP14 inhibitors that have been developed from the compound library using small-molecule microarray technology (Peng, B.; Thorsell, A.-G.; Karlberg, T.; Schueler, H.; Yao, S. Q., Small molecule microarray based discovery of PARP14 inhibitors. Angew. Chem., Int. Ed. 2017, 56 (1), 248-253.), and they can interact with the primary and secondary binding pockets of PARP14 (FIG. 2).

GeA-69, a compound containing a fused ring-indole structure, is a selective PARP14 allosteric inhibitor targeting a Macrodomain 2 (MD2), K_{d}=2.1 μM. Inspired by the above molecular design strategy of linking different structural fragments through linking groups, a bis-heterocyclic structural system containing a 1H-indole ring and a nitrogen and sulfur-containing five-membered heterocycle connected via a linker has been designed and synthesized herein to fit two binding sites of PARP14 (Site A and Site B, FIG. 3).

### SUMMARY

An object of the disclosure is to provide a novel PARP14 inhibitor with excellent site-specificity, high potency and low toxicity.

In order to solve the general problem of low selectivity of the available PARP inhibitors, it is desired to design an inhibitor that can act simultaneously on the sites A and B by combining different active fragments via a suitable linker based on the combination principles (FIGS. 2 and 3).

Based on the combination principle, pharmacophore model and characteristics of the PARP14 active site, an aromatic indole and a 1,3-thiazolidin-4-one five-membered heterocycle are selected herein as the basic active scaffolds, which are fused into a molecule through the linking groups with various length. The target compounds (TM1-TM6) were designed by introducing various substituents at different sites of the basic parent nucleus of 1H-indol-2-one and 1,3-thiazolidin-4-one to adjust the molecular size of the inhibitors. Generally, the selection of a linker with more potential hydrogen bond donors is conducive to improving the inhibitory activity against the target. However, a hydrazono group with a hydrogen bond acceptor is selected herein as the linking group to satisfy the spatial location and distance requirements between various binding sites in the binding cavity of the PARP14 receptor. Further, through introducing more substituent groups, these small molecules can stretch to multiple sites (Site A and Site B) of the target proteins to produce a more sufficient interaction, so as to discover a PARP14 inhibitor candidate with stable physicochemical property, high activity, and strong selectivity. The design idea of the target compound provided herein is shown in FIG. 3.

Technical solutions of the present disclosure are described as follows.

In a first aspect, this application provides a novel PARP14 inhibitor, which is an indolone-substituted 1,3-thiazolidinone derivative, as shown in the following formula:

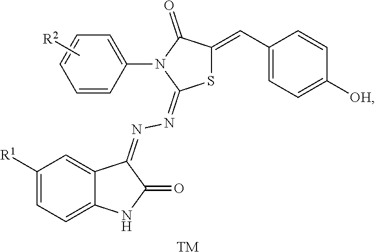

wherein R¹ is a halogen electron-withdrawing or an alkyl electron-donating group; and
R² is a 3-position or 4-position substituent selected from the group consisting of an electron-withdrawing group and an electron-donating group.

In some embodiments, the arylidene-indolone-3-substituted thiazolidinone derivative is selected from the group consisting of TM1 with R¹ being CH₃ and R² being 3-CF₃, TM2 with R¹ being F and R² being 3-Cl, TM3 with R¹ being CH₃ and R² being 3-Cl, TM4 with R¹ being F and R² being 3-CF₃, TM5 with R¹ being Cl and R² being 4-F, TM6 with R¹ being Br and R² being 4-C(CH₃)₃.

In a second aspect, this application provides a pharmaceutical composition, comprising:
the arylidene-indolone-3-substituted thiazolidinone derivative herein, or a pharmaceutically-acceptable salt, a hydrate, a solvate, a polycrystal, a eutectic, a precursor and a derivative thereof.

In some embodiments, the pharmaceutically-acceptable salt is selected from the group consisting of hydrochloride, hydrobromide, sulfate, phosphate, acetate, methanesulfonate, p-toluenesulfonate, tartrate, citrate, fumarate and malate.

In a third aspect, this application provides a method of preparing the arylidene-indolone-3-substituted thiazolidinone derivative, comprising:
(1) reacting 1.0-1.1 equiv. of a substituted aromatic amine (1) with 1.1-1.2 equiv. of chloral hydrate and 3.0-3.3 equiv. of hydroxylamine hydrochloride followed by silica gel column chromatography to collect an intermediate (2), with yields ranging from 62.6% to 87.6%;
(2) subjecting the intermediate (2) to cyclization in the presence of concentrated sulphuric acid to obtain 5-substituted isatin (3), with yields ranging from 70.8% to 98.3%;
(3) reacting 1.0-1.1 equiv. of a substituted aniline (1.1) with 1.8-2.0 equiv. of CS₂ (4) under an alkaline condition followed by separation and purification to collect a thioglycolate intermediate; subjecting the thioglycolate intermediate to desulfurization in the presence of 1.0-1.1 equiv. of methyl chloroformate and silica gel column chromatography to obtain a substituted aryl isothiocyanate (5), with yields ranging from 40.6% to 85.5%;
(4) subjecting the substituted aryl isothiocyanate (5) to hydrazinolysis in the presence of hydrazine hydrate (80 wt. %) to obtain a N-aryl-substituted thiosemicarbazide intermediate (6), with yields ranging from 58.2% to 87.8%;
(5) subjecting 1.0-1.1 equiv. of the 5-substituted isatin (3) and 1.0-1.1 equiv. of the N-aryl-substituted thiosemicarbazide intermediate (6) to intermolecular dehydration condensation in ethanol under the catalysis of concentrated sulphuric acid to obtain a disubstituted thiosemicarbazone intermediate (7), with yields ranging from 58.3% to 87.8%;
(6) subjecting 1.0-1.1 equiv. of the disubstituted thiosemicarbazone intermediate (7) and 1.0-1.1 equiv. of ethyl 2-chloroacetate to cyclization reaction under the catalysis of anhydrous sodium acetate in an equimolar ratio to obtain an intermediate (8), with yields ranging from 72.1% to 97.7%; and
(7) subjecting 1.0-1.1 equiv. of the intermediate (8) and 1.0-1.1 equiv. of p-hydroxybenzaldehyde (9) to a Knoevenagel condensation reaction under the catalysis of 1% by mole of anhydrous piperidine, and separation and purification to obtain the arylidene-indolone-3-substituted thiazolidinone derivative;
as shown in the following synthesis route:

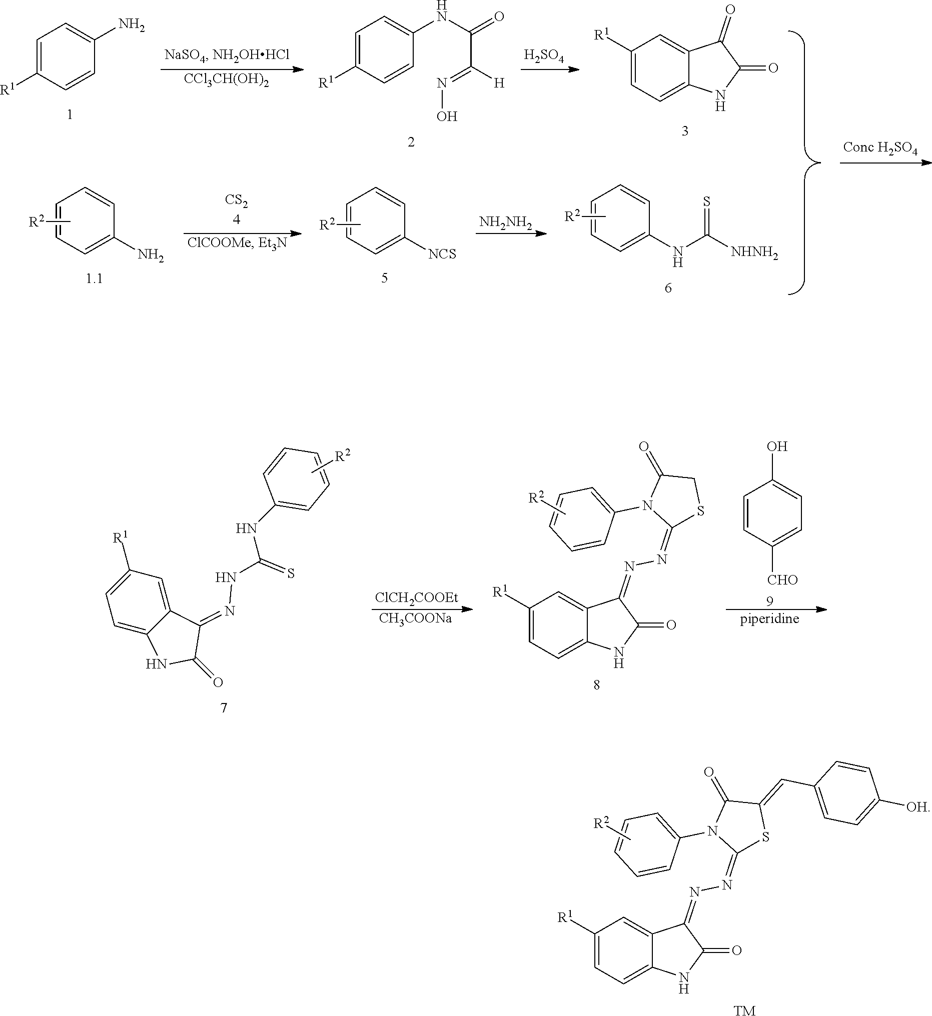

In some embodiments, the arylidene-indolone-3-substituted thiazolidinone derivative is selected from the group consisting of TM1 with R¹ being CH₃ and R² being 3-CF₃, TM2 with R¹ being F and R² being 3-Cl, TM3 with R¹ being CH₃ and R² being 3-Cl, TM4 with R¹ being F and R² being 3-CF₃, TM5 with R¹ being Cl and R² being 4-F, and TM6 with R¹ being Br and R² being 4-C(CH₃)₃;
as shown in the following formulas:

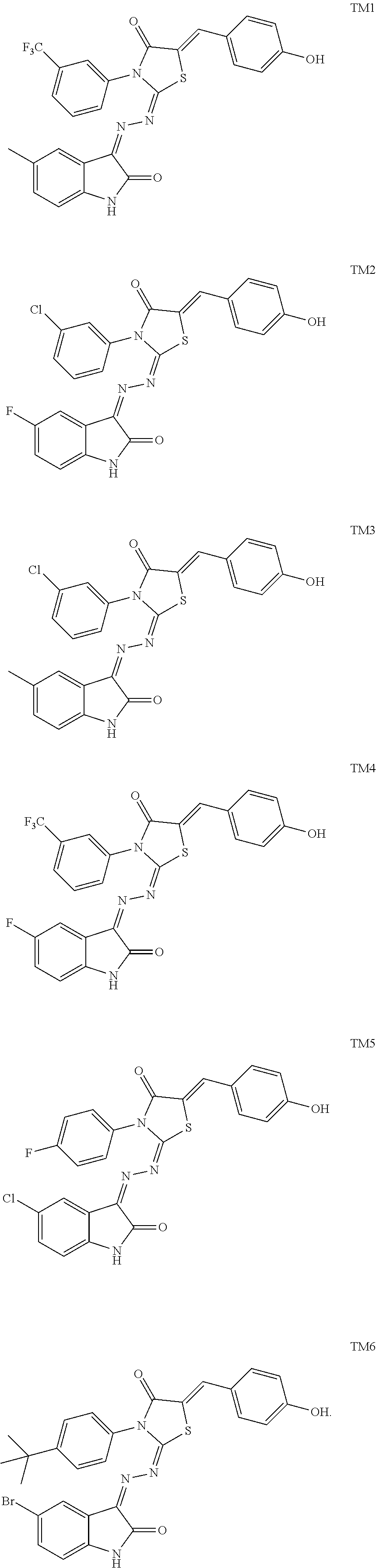 [SMILES: Cc1ccc2c(c1)/C(=N/N=C1/S/C(=C\c3ccc(O)cc3)C(=O)N1c1cccc(C(F)(F)F)c1)C(=O)N2 ; O=C1Nc2ccc(F)cc2/C1=N/N=C1/S/C(=C\c2ccc(O)cc2)C(=O)N1c1cccc(Cl)c1 ; Cc1ccc2c(c1)/C(=N/N=C1/S/C(=C\c3ccc(O)cc3)C(=O)N1c1cccc(Cl)c1)C(=O)N2 ; O=C1Nc2ccc(F)cc2/C1=N/N=C1/S/C(=C\c2ccc(O)cc2)C(=O)N1c1cccc(C(F)(F)F)c1 ; O=C1Nc2ccc(Cl)cc2/C1=N/N=C1/S/C(=C\c2ccc(O)cc2)C(=O)N1c1ccc(F)cc1 ; CC(C)(C)c1ccc(N2C(=O)/C(=C/c3ccc(O)cc3)S/C2=N/N=C2\C(=O)Nc3ccc(Br)cc32)cc1]

The indole alkaloid-substituted 1,3-thiazolidinone derivative designed and synthesized herein is demonstrated by nuclear magnetic resonance (NMR) and mass spectrometry to possess a novel structural. The thermal shift assay between the small-molecule compound provided herein and the PARP14 protein macromolecule is performed through the in vitro enzyme activity test, and the results show that all six target compounds will result in obvious thermal change (Δ_{TM} (temperature alteration) is greater than three) after bound to the target PARP14. This strong positive value generally indicates that there is stronger interaction of the compound with the target protein, and the compound could be used as a lead compound for PARP14 inhibitors.

### BRIEF DESCRIPTION OF THE DRAWINGS

FIG. 1 schematically shows a binding domain that is closely related to a PARP1 inhibitor.

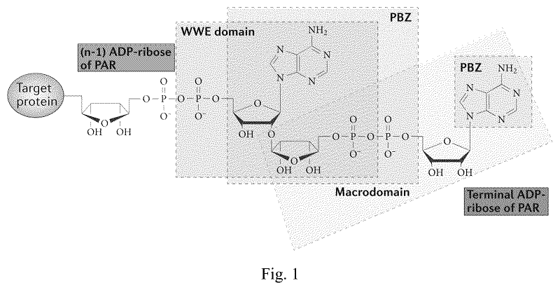

FIG. 2 schematically shows the existing PARP14 inhibitors and potential active sites of PARP14.

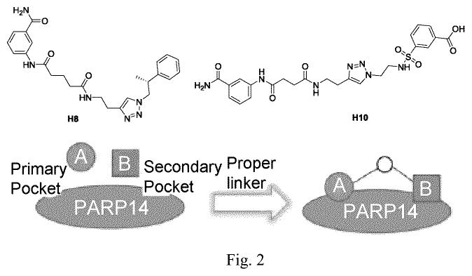

FIG. 3 schematically shows a design idea of the target PARP14 inhibitor.

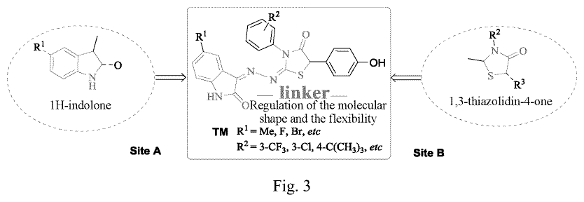

FIG. 4A schematically illustrates an interaction between the whole enzyme of A-chain of PARP14 and TM1 molecule; and

FIG. 4B schematically shows the interaction between the TM1 molecule and a catalytic structural domain of the PARP14 (the three hydrogen bonds are indicated by dashed lines).

### DETAILED DESCRIPTION OF EMBODIMENTS

The present disclosure will be further described below in conjunction with embodiments.

The embodiments include synthesis of relevant intermediates and target compound, evaluation of inhibitory activity against PARP14 and analysis of relevant data and structure-activity relationship.

### Example 1

### Synthesis of N-Hydroxamic Acetyl-Substituted Aniline Intermediates 2a-2c

To a clean 500 mL single-necked flask were added chloral hydrate (9.0 g, 55.0 mmol) and water (240 mL). The reaction mixture was stirred, sequentially added with sodium sulfate anhydrous (130 g), a substituted aniline (1a-1c, 50.0 mmol), a hydrochloric acid solution (2.2 mL HCl+10.0 mL water), hydroxylamine hydrochloride (10.4 g, 150.0 mmol), heated to 65° C. and reacted for 2 h. After the reaction was completed, the reaction mixture was immediately filtered to collect a solid crude product. The solid crude product corresponding to the substituted aniline 1a was subjected to column purification (eluent: PE:EA=5:1˜3:1) to obtain a yellow solid 2a (60.5% yield, m.p. 54.9˜155.7° C.); the solid crude product corresponding to the substituted aniline 1b was subjected to column purification (eluent: PE:EA=3:1˜2:1) to obtain a light yellow solid 2b (85.6% yield, m.p. 158.5˜160.1° C.); and the solid crude product corresponding to the substituted aniline 1c was subjected to column purification (eluent: PE:EA=5:1˜3:1) to obtain a yellow solid 2c (78.3% yield, m.p. 166.8˜168.3° C.).

### Example 2

### Synthesis of 5-Substituted Isatin Intermediates 3a-3c

Concentrated sulfuric acid (24.0 mL) was added to a clean 150 mL three-necked flask, and heated to 50° C., to which the intermediate (2a-2c, 30.0 mmol) was slowly added and the color of the solution slowly deepened and turned black as the intermediate was added. The reaction mixture was heated to 80° C., reacted for 20 min, slowly added with crushed ice (100 g) (the system was reddish brown), and subjected to standing and vacuum filtration to obtain a filter residue. The filter residue was washed to neutrality, dissolved in 90 mL of 10% NaOH, adjusted to pH 4 with concentrated hydrochloric acid, and subjected to vacuum filtration to obtain a filtrate. The filtrate was adjusted to pH 2 with concentrated hydrochloric acid, accompanied by the precipitation of a lot of brick-red solids, and subjected to vacuum filtration and drying to obtain reddish-brown solid 3a (85.9% yield, 185.3˜186.8° C.), 3b (88.6% yield, 219.1˜220.8° C.), or 3c (80.3% yield, 225.8˜227.1° C.).

### Example 3

### Synthesis of Aryl Isothiocyanate Intermediates 5a-5c

Each substituted aniline (1.1a-1.1c, 50.0 mmol) was weighed and placed in a clean 100 mL three-necked flask, to which ethyl ether (15.0 mL), CS₂ 4 (3.6 mL, 90.0 mmol), and triethylamine (7.2 mL) were added. The reaction mixture was reacted at 25-30° C. for 12 h and filtered under vacuum to collect a filter cake, which was washed with anhydrous ethyl ether (30.0 mL) to obtain a powdered solid. The powdered solid was dried naturally in air for 10 min to allow complete volatilization of the residual ethyl ether, and then transferred to a 100 mL clean three-necked flask, to which chloroform (50.0 mL) was added to form a homogeneous system. The system was added with triethylamine (7.2 mL), cooled to 0° C. or below in an ice-salt bath, and dropwise added with methyl chloroformate (3.9 mL, 50.0 mmol) under stirring, where the system temperature was controlled to be 5° C. or below during the dropwise addition. The reaction system was reacted at 29-30° C. for 1 h, where the reaction was monitored by thin-layer chromatography (TLC). When the reaction was completed, the reaction mixture was mixed with silica gel and separated by column chromatography (petroleum ether system) to obtain a colorless oily product or a white solid, namely, intermediates 5a-5c (Table 1).

TABLE 1
Aryl isothiocyanate intermediates 5a-5c

| Compound | Yield (%) | Appearance | Melting point (° C.) |
|---|---|---|---|
| 5a | 65.4 | Colorless, oily | — |
| 5b | 80.3 | Colorless, oily | — |
| 5c | 87.6 | White, solid | 37-38 |

### Example 4

### Synthesis of N-Substituted Thiosemicarbazide Intermediates 6a-6c

Individual aryl isothiocyanate intermediates (5a-5c, 2.00 mmol) were respectively added to a clean 50 mL single-necked flask, and dissolved with 20 mL of isopropanol. The reaction mixture was dropwise added with hydrazine hydrate (85%, 2.40 mmol) under stirring (a large amount of white precipitate was immediately produced), stirred at room temperature for 30 min, and filtered to obtain a filter residue, which was washed three times with isopropanol to obtain N-substituted thiosemicarbazide intermediates 6a-6c, respectively (Table 2).

TABLE 2
N-substituted thiosemicarbazide intermediates 6a-6c

| | | Melting point |
|---|---|---|
| Compound | Yield (%) | (° C.) |
| 6a | 89.8 | 118.5-120.6 |
| 6b | 77.2 | 125.5-127.3 |
| 6c | 63.0 | 137.3-138.9 |

### Example 5

### Synthesis of Disubstituted-Thiosemicarbazone Intermediates 7a-7f

Individual 5-substituted isatin intermediates (3a-3c, 3.50 mmol and 95% ethanol (30.0 mL) were added to a clean 100 mL single-necked flask, respectively, to which individual N-substituted thiosemicarbazide intermediates (6a-6c, 3.50 mmol) were added with stirring. When the mixture was homogeneous, a drop of concentrated sulfuric acid was added to the reaction mixture, and the temperature was increased to reflux for 5 h gradually and monitored by TLC. When the reaction was complete, the mixture was cooled to room temperature (precipitate was produced), and subjected to vacuum filtration to obtain a filter residue, which was washed with cold ethanol absolute to obtain an orange-red solid as the desired intermediate, namely, intermediates 7a-7f (Table 3).

TABLE 3
Disubstituted-thiosemicarbazone intermediates 7a-7f

| | | | Melting |
|---|---|---|---|
| | | Yield | point |
| Compound | Name | (%) | (° C.) |
| 7a | 5-methyl-2-oxo-1H-indole-3-imino- | 64.9% | 266.6- |
| | 4-(3-trifluoromethylphenyl)thiourea | | 267.9 |
| 7b | 5-fluoro-2-oxo-1H-indole-3-imino- | 58.2% | 255.1- |
| | 4-(3-chlorophenyl)thiourea | | |
| | | | 257.6 |
| 7c | 5-methyl-2-oxo-1H-indole-3-imino- | 80.9% | 249.1- |
| | 4-(3-chlorophenyl)thiourea | | |
| | | | 250.0 |
| 7d | 5-fluoro-2-oxo-1H-indole-3-imino- | 85.2% | 244.8- |
| | 4-(3-trifluoromethylphenyl)thiourea | | |
| | | | 246.9 |
| 7e | 5-chloro-2-oxo-1H-indole-3-imino- | 73.6% | 282.5- |
| | 4-(4-fluorophenyl)thiourea | | 283.7 |
| 7f | 5-bromo-2-oxo-1H-indole-3-imino- | 71.8% | 261.6- |
| | 4-(4-tert-butylphenyl)thiourea | | 263.5 |

### Example 6

### Synthesis of Disubstituted-1,3-Thiazolidin-2-One Intermediates 8a-8f

### (1) Synthesis of 5-methyl-3-(2-(3-(3-chlorophenyl)-4-oxothiazolidine-2-diyl) hydrazono)-1H-indol-2-one (8a)

Intermediate 7a (0.38 g, 1.00 mmol) was weighed and placed in a clean 50 mL single-necked flask, to which 95% ethanol (20.0 mL) was added. Anhydrous sodium acetate (0.34 g, 4.00 mmol) was then added under stirring, followed by the dropwise addition of ethyl chloroacetate (0.24 mL, 2.00 mmol). The reaction mixture was gradually heated to 78° C. and refluxed for about 5 h. When the reaction was completed, the mixture was cooled to room temperature, followed by addition of an appropriate amount of water to dilute the mixture (precipitate was produced), and subjected to vacuum filtration to obtain a filter residue, which was washed with cold ethanol absolute to obtain a yellow solid, and dried to obtain a red solid, which was a pure product, namely, intermediate 8a (0.32 g, 76.1% yield), m.p.>300° C.

¹H NMR (400 MHz, DMSO-d₆) δ: 10.60 (s, 1H, indole-NH), 7.99 (m, 2H, 3-N—Ar—H), 7.89 (d, J=8.2 Hz, 2H, 3-N—Ar—H), 7.06 (m, 1H, indole-6-H), 6.94 (s, 1H, indole-4-H), 6.69 (d, J=7.9 Hz, 1H, indole-7-H), 4.21 (s, 2H, thiazolidine —CH₂—), 1.91 (s, 3H, indole-5-CH₃).

### (2) Synthesis of 5-fluoro-3-(2-(3-(3-chlorophenyl)-4-oxothiazolidine-2-diyl) hydrazono)-1H-indol-2-one (8b)

Intermediate 7b (1.04 g, 3.00 mmol) was weighed and placed in a clean 250 mL single-necked flask, to which 95% ethanol (60.0 mL) was added. Anhydrous sodium acetate (0.98 g, 12.0 mmol) was then added under stirring, followed by the dropwise addition of ethyl chloroacetate (0.7 mL, 6.00 mmol). The reaction mixture was gradually heated to 78° C. and refluxed for about 5 h. When the reaction was completed, the mixture was cooled to room temperature, followed by addition of an appropriate amount of water to dilute the mixture (precipitate was produced), and subjected to vacuum filtration to obtain a filter residue, which was washed with cold ethanol absolute to obtain a yellow solid, and dried to obtain a pure product, namely, intermediate 8b (1.13 g, 97.4% yield), m.p.>300° C.

¹H NMR (400 MHz, DMSO-d₆) δ: 10.74 (s, 1H, indole-NH), 7.70 (d, J=1.4 Hz, 1H, 3-N—Ar-4-H), 7.66 (d, J=1.2 Hz, 1H, 3-N—Ar-2-H), 7.64 (t, J=5.2 Hz, 1H, 3-N—Ar-6-H), 7.52 (m, 1H, 3-N—Ar-5-H), 7.14 (td, J=9.1, 2.8 Hz, 1H, indole-6-H), 6.98 (dd, J=8.7, 2.8 Hz, 1H, indole-4-H), 6.81 (dd, J=8.6, 4.3 Hz, 1H, indole-7-H), 4.22 (s, 2H, thiazolidine-CH₂—).

### (3) Synthesis of 5-methyl-3-(2-(3-(3-chlorophenyl)-4-oxothiazolidine-2-diyl) hydrazono)-1H-indol-2-one (8c)

Intermediate 7c (0.45 g, 1.30 mmol) was weighed and placed in a clean 50 mL single-necked flask, to which 95% ethanol (25.0 mL) was added. Anhydrous sodium acetate (0.43 g, 5.20 mmol) was then added under stirring, followed by the dropwise addition of ethyl chloroacetate (0.32 mL, 2.60 mmol). The reaction mixture was gradually heated to 78° C. and refluxed for about 5 h. And the mixture was cooled to room temperature, followed by addition of an appropriate amount of water to dilute the mixture (precipitate was produced), and subjected to vacuum filtration to obtain a filter residue, which was washed with cold ethanol absolute to obtain a tangerine solid, and dried to obtain a pure product, namely, intermediate 8c (0.48 g, 96.0% yield), m.p.>300° C.

¹H NMR (400 MHz, DMSO-d₆) δ: 10.60 (s, 1H, indole-NH), 7.70 (s, 1H, 3-N-2-Ar—H), 7.67 (dd, J=4.0, 1.3 Hz, 2H, 3-N—Ar-4, 6-H), 7.52 (m, 1H, 3-N-5-Ar—H), 7.08 (d, J=5.0 Hz, 2H, indole-4, 6-H), 6.70 (d, J=8.4 Hz, 1H, indole-7-H), 4.21 (s, 2H, thiazolidine —CH₂—), 2.02 (s, 3H, indole-5-CH₃).

### (4) Synthesis of 5-fluoro-3-(2-(3-(3-trifluoromethylphenyl)-4-oxothiazo-lidine-2-diyl)hydrazono)-1H-indol-2-one (8d)

Intermediate 7d (0.38 g, 1.00 mmol) was weighed and placed in a clean 50 mL single-necked flask, to which 95% ethanol (20.0 mL) was added. Anhydrous sodium acetate (0.34 g, 4.00 mmol) was then added under stirring, followed by the dropwise addition of ethyl chloroacetate (0.24 mL, 2.00 mmol). The reaction mixture was gradually heated to 78° C. and refluxed for about 5 h. When the reaction was completed, the mixture was cooled to room temperature, followed by addition of an appropriate amount of water to dilute the mixture (precipitate was produced), and subjected to vacuum filtration to obtain a filter residue, which was washed with cold ethanol absolute to obtain a khaki solid and dried to obtain a pure product, namely, intermediate 8d (0.28 g, 66.7% yield), m.p.>300° C.

¹H NMR (400 MHz, DMSO-d₆) δ: 10.74 (s, 1H, indole-NH), 8.01 (s, 1H, 3-N—Ar-2-H), 7.96 (m, 1H, 3-N—Ar-4-H), 7.86 (dd, J=4.9, 1.4 Hz, 2H, 3-N—Ar-5,6-H), 7.12 (td, J=9.1, 2.8 Hz, 1H, indole-6-H), 6.82 (ddd, J=12.9, 8.6, 3.5 Hz, 2H, indole-4,7-H), 4.23 (s, 2H, thiazolidine-CH₂—).

### (5) Synthesis of 5-chloro-3-(2-(3-(4-fluorophenyl)-4-oxothiazolidine-2-diyl) hydrazono)-1H-indol-2-one (8e)

Intermediate 7e (0.52 g, 1.50 mmol) was weighed and placed in a clean 100 mL single-necked flask, to which 95% ethanol (50.0 mL) was added. Anhydrous sodium acetate (0.50 g, 6.00 mmol) was then added under stirring, followed by the dropwise addition of ethyl chloroacetate (0.36 mL, 3.00 mmol). The reaction mixture was gradually heated to 78° C. and refluxed for about 5 h. When the reaction was completed, the mixture was cooled to room temperature, followed by addition of an appropriate amount of water to dilute the mixture, and subjected the mixture to stand overnight and vacuum filtration to obtain a filter residue, which was washed with cold ethanol absolute to obtain a tangerine solid and dried to obtain a pure product, namely, intermediate 8e (0.54 g, 93.1% yield), m.p.>300° C.

¹H NMR (400 MHz, DMSO-d₆) δ: 10.82 (s, 1H, indole-NH), 7.57 (dd, J=8.9, 5.0 Hz, 2H, 3-N—Ar-3, 5-H), 7.45 (t, J=8.8 Hz, 2H, 3-N—Ar-2, 6-H), 7.31 (dd, J=8.3, 2.3 Hz, 1H, indole-6-H), 7.20 (d, J=2.2 Hz, 1H, indole-4-H), 6.81 (d, J=8.3 Hz, 1H, indole-7-H), 4.23 (s, 2H, thiazolidine-CH₂—).

### (6) Synthesis of 5-bromo-3-(2-(3-(4-tert-butylphenyl)-4-oxothiazolidine-2-diyl)hydrazono)-1H-indol-2-one (8f)

Intermediate 7f (1.61 g, 3.60 mmol) was weighed and placed in a clean 100 mL single-necked flask, to which 95% ethanol absolute (50.0 mL) was added. Anhydrous sodium acetate (1.18 g, 14.4 mmol) was then added under stirring, followed by the dropwise addition of ethyl chloroacetate (0.49 mL, 4.00 mmol). The reaction mixture was gradually heated to 78° C. and refluxed for about 8 h. When the reaction was completed, the mixture was cooled to room temperature, followed by addition of an appropriate amount of water to dilute the mixture (precipitate was produced), and subjected to vacuum filtration to obtain a filter residue, which was washed with cold ethanol absolute and dried to obtain an orange red solid pure product, namely, intermediate 8f (1.68 g, 98.8% yield) with m.p.>300° C. (eluent: PE:EA=1:2, Rf=0.2).

¹H NMR (400 MHz, DMSO-d₆) δ: 11.00 (s, 1H, indole-NH), 7.62 (d, J=8.6 Hz, 2H, 3-N—Ar-2, 6-H), 7.55 (d, J=2.1 Hz, 1H, 3-N—Ar-5-H), 7.43 (dd, J=8.3, 2.1 Hz, 1H, 3-N—Ar-3-H), 7.41 (s, 1H, 3-N—Ar-4-H), 7.39 (s, 1H, indole-Ar-4-H), 6.80 (d, J=8.3 Hz, 1H, indole-Ar-6-H), 4.22 (s, 2H, thiazolidine-CH₂—), 1.35 (s, 9H, 3-N—Ar-4-C(CH₃)₃). 13C NMR (100 MHz, DMSO-d₆) δ: 174.54 (s), 172.70 (s), 164.70 (s), 151.90 (s), 148.34 (s), 143.70 (s), 135.42 (s), 132.51 (s), 130.65 (s), 127.70 (s), 126.64 (s), 118.74 (s), 113.64 (s), 112.92 (s), 35.06 (s), 33.38 (s), 31.60 (s).

### Example 7

### Synthesis of the Target Compounds TM1-TM6

### (1) Synthesis of 3-(2-(5-(4-hydroxybenzylidene)-3-(3-trifluoromethylphenyl)-4-oxothiazolidine-2-diyl)hydrazono)-5-methyl-1H-indol-2-one (TM1)

Intermediate 8a (0.21 g, 0.50 mmol) and 4-hydroxybenzaldehyde 9 (0.067 g, 0.55 mmol) were weighed and placed in a clean 50 mL flask, to which ethanol absolute (15.0 mL) was added, followed by addition of anhydrous piperidine (0.1 mL) under stirring. The reaction mixture was heated to reflux for 6 h. When the reaction was completed, the reaction mixture was cooled to room temperature (a large amount of red precipitate was produced), and subjected to vacuum filtration to obtain a filter residue, which was washed with a small amount of ethanol and dried to obtain a red powdered solid, namely, TM1 (0.237 g, 90.8% yield), m.p.>300° C.

¹H NMR (400 MHz, DMSO-d₆) δ: 10.64 (s, 1H, indole-NH), 10.42 (s, 1H, thiazolidine-5-Ar—OH), 8.18 (s, 1H, 3-N—Ar-2-H), 8.01 (d, J=7.7 Hz, 2H, 3-N—Ar—H), 7.92 (m, 1H, 3-N—Ar—H), 7.79 (s, 1H, thiazolidine-5-=CH), 7.62 (d, J=8.1 Hz, 2H, thiazolidine-5-Ar-2, 6-H), 7.03 (dd, J=24.6, 8.4 Hz, 4H, thiazolidine-5-Ar-3, 5-H, indole-4, 6-H), 6.69 (d, J=7.8 Hz, 1H, indole-7-H), 1.91 (s, 3H, indole-5-CH₃).

### (2) Synthesis of 3-(2-(5-(4-hydroxybenzylidene)-3-(3-chlorophenyl)-4-oxothiazolidine-2-diyl) hydrazono)-5-fluoro-1H-indol-2-one (TM2)

Intermediate 8b (0.23 g, 0.60 mmol) and 4-hydroxybenzaldehyde 9 (0.08 g, 0.66 mmol) were weighed and placed in a clean 50 mL flask, to which ethanol absolute (10.0 mL) was added, followed by addition of anhydrous piperidine (0.12 mL) under stirring. The reaction was heated to reflux for 5 h. When the reaction was completed, the mixture was cooled to room temperature (a large amount of red precipitate was produced), and subjected to vacuum filtration to obtain a filter residue, which was washed with a small amount of ethanol and dried to obtain a red powdered solid, namely, TM2 (0.245 g, 82.8% yield), m.p.>300° C.

¹H NMR (400 MHz, DMSO-d₆) δ: 10.77 (s, 1H, indole-NH), 10.41 (s, 1H, thiazolidine-5-Ar—OH), 7.85 (s, 1H, thiazolidine-5-=CH), 7.82 (s, 1H, 3-N—Ar-2-H), 7.68 (m, 2H, thiazolidine-5-Ar-2, 6-H), 7.63 (m, 3H, 3-N—Ar—H), 7.13 (m, 1H, indole-4-H), 7.05 (dd, J=8.6, 2.6 Hz, 1H, indole-6-H), 7.00 (d, J=8.6 Hz, 2H, thiazolidine-5-Ar-3, 5-H), 6.81 (dd, J=8.5, 4.3 Hz, 1H, indole-7-H).

### (3) Synthesis of 3-(2-(5-(4-hydroxybenzylidene)-3-(3-chlorophenyl)-4-oxothiazolidine-2-diyl) hydrazono)-5-methyl-1H-indol-2-one (TM3)

Intermediate 8c (0.23 g, 0.60 mmol) and compound 4-hydroxybenzaldehyde 9 (0.08 g, 0.66 mmol) were weighed and placed in a clean 50 mL flask, to which ethanol absolute (15.0 mL) was added, followed by addition of anhydrous piperidine (0.15 mL) under stirring. The reaction mixture was heated to reflux for 6 h. When the reaction was completed, the reaction mixture was cooled to room temperature (a large amount of red precipitate was produced), and subjected to vacuum filtration to obtain a filter residue, which was washed with a small amount of ethanol and dried to obtain a red powdered solid, namely, TM3 (0.278 g, 94.9% yield), m.p.>300° C.

¹H NMR (400 MHz, DMSO-d₆) δ: 10.65 (s, 1H, indole-NH), 10.39 (s, 1H, thiazolidine-5-Ar—OH), 7.86 (s, 1H, 3-N—Ar-2-H), 7.79 (s, 1H, thiazolidine-5-=CH), 7.70 (d, J=5.2 Hz, 2H, 3-N—Ar-4, 6-H), 7.65 (m, 1H, 3-N—Ar-5-H), 7.62 (d, J=8.7 Hz, 2H, thiazolidine-5-Ar-2, 6-H), 7.15 (s, 1H, indole-4-H), 7.09 (d, J=8.0 Hz, 1H, indole-6-H), 7.00 (d, J=8.6 Hz, 2H, thiazolidine-5-Ar-2, 6-H), 6.70 (d, J=7.9 Hz, 1H, indole-7-H), 2.01 (s, 3H, indole-5-CH₃).

### (4) Synthesis of 3-(2-(5-(4-hydroxybenzylidene)-3-(3-trifluoromethylphenyl)-4-oxothiazolidine-2-diyl) hydrazono)-5-fluoro-1H-indol-2-one (TM4)

Intermediate 8d (0.19 g, 0.45 mmol) and 4-hydroxybenzaldehyde 9 (0.06 g, 0.50 mmol) were weighed and placed in a clean 50 mL flask, to which ethanol absolute (15.0 mL) was added, followed by addition of anhydrous piperidine (0.1 mL) under stirring. The reaction was heated to reflux for 6 h. When the reaction was completed, the reaction mixture was cooled to room temperature (a large amount of red precipitate was produced), and subjected to vacuum filtration to obtain a filter residue, which was washed with a small amount of ethanol and dried to obtain a red powdered solid, namely, TM4 (0.193 g, 81.4% yield), m.p.>300° C.

¹H NMR (400 MHz, DMSO-d₆) δ: 10.78 (s, 1H), 7.53 (tdd, 12H).

### (5) Synthesis of 3-(2-(5-(4-hydroxybenzylidene)-3-(4-fluorophenyl)-4-oxothiazolidine-2-diyl) hydrazono)-5-chloro-1H-indol-2-one (TM5)

Intermediate 8e (0.272 g, 0.70 mmol) and 4-hydroxybenzaldehyde 9 (0.095 g, 0.77 mmol) were weighed and placed in a clean 50 mL flask, to which ethanol absolute (15.0 mL) was added, followed by addition of anhydrous piperidine (0.15 mL) under stirring. The reaction was heated to reflux for 6 h. When the reaction was completed, the mixture was cooled to room temperature (a large amount of red precipitate was produced), and subjected to vacuum filtration to obtain a filter residue, which was washed with a small amount of ethanol and dried to obtain a red powdered solid, namely, TM5 (0.294 g, 85.2% yield), m.p.>300° C.

¹H NMR (400 MHz, DMSO-d₆) δ: 10.86 (s, 1H, indole-NH), 10.40 (s, 1H, thiazolidine-5-Ar—OH), 7.81 (s, 1H, thiazolidine-5-=CH), 7.70 (dd, J=8.8, 5.0 Hz, 2H, 3-N—Ar-3, 5-H), 7.62 (d, J=8.7 Hz, 2H, thiazolidine-5-Ar-2, 6-H), 7.48 (t, J=8.8 Hz, 2H, 3-N—Ar-2, 6-H), 7.31 (dd, J=8.3, 2.2 Hz, 1H, indole-6-H), 7.26 (d, J=2.1 Hz, 1H, indole-4-H), 6.99 (d, J=8.6 Hz, 2H, thiazolidine-5-Ar-3, 5-H), 6.81 (d, J=8.3 Hz, 1H, indole-7-H).

### (6) Synthesis of 3-(2-(5-(4-hydroxybenzylidene)-3-(4-tert-butylphenyl)-4-oxothiazolidine-2-diyl) hydrazono)-5-bromo-1H-indole-2-one (TM6)

Intermediate 8f (0.471 g, 1.00 mmol) and 4-hydroxybenzaldehyde 9 (0.122 g, 1.00 mmol) were weighed and placed in a clean 50 mL flask, to which ethanol absolute (15.0 mL) was added, followed by addition of anhydrous piperidine (0.2 mL) under stirring. The reaction mixture was heated to reflux for 7 h. When the reaction was completed, the reaction mixture was cooled to room temperature (a large amount of red precipitate was produced), and subjected to vacuum filtration to obtain a filter residue, which was washed with a small amount of ethanol and dried to obtain an orange powdered solid, namely, TM6 (0.456 g, 79.3% yield) with m.p.>300° C., (eluent: PE:EA=1:3, Rf=0.2).

¹H NMR (400 MHz, DMSO-d₆) δ: 10.95 (s, 1H, indole-NH), 7.78 (s, 1H, thiazolidine-5=CH), 7.64 (s, 1H, 3-N—Ar-2-H), 7.62 (s, 1H, 3-N—Ar-6-H), 7.61 (s, 1H, 3-N—Ar-3-H), 7.59 (d, J=1.9 Hz, 1H, 3-N—Ar-5-H), 7.50 (d, J=8.5 Hz, 2H, thiazolidine-5-Ar-3, 6-H), 7.42 (dd, J=8.3, 2.1 Hz, 2H, thiazolidine-5-Ar-3, 5-H), 7.01 (d, J=8.6 Hz, 2H), 6.78 (d, J=8.3 Hz, 1H, indole-6-H), 1.36 (s, 9H, 3-N—Ar-4-C(CH₃)₂). ¹³C NMR (100 MHz, DMSO-d6) δ: 168.78 (s), 166.36 (s), 164.58 (s), 160.67 (s), 152.00 (s), 148.82 (s), 143.90 (s), 135.67 (s), 133.54 (s), 133.13 (s), 132.35 (s), 130.71 (s), 127.76 (s), 126.59 (s), 124.63 (s), 118.72 (s), 116.99 (s), 116.46 (s), 113.74 (s), 112.96 (s), 35.07 (s), 31.60 (s).

### Example 8

### Evaluation of the Inhibitory Activity of Target Compounds (TM1-TM6) Against PARP14 Enzyme

The binding between all the target compounds and the PARP-1 target was tested by thermal shift analysis (TSA) to preliminarily evaluate their potential bioactivities. The experiment was designed as follows: control sample: fluorescent dye+dimethyl sulfoxide (DMSO)+buffer; reference sample: protein+fluorescent dye+DMSO+buffer; and experimental sample: protein+fluorescent dye+inhibitor+buffer. The experimental results were observed by using a real-time quantitative polymerase chain reaction (qPCR) system, and the data analysis was performed by using Protein Thermal Shift™ software.

The principle of the TSA test was described as follows. Proteins were sensitive to external temperature, and therefore, heating will induce the protein denaturation. The thermal stability of proteins referred to their ability to maintain biological activity under the exposure of elevated temperature and other factors, which was an important indicator of protein stability. TSA was a method used to assess the thermal stability of proteins. In TSA, proteins were gradually warmed as the external temperature increased, and when the temperature exceeded the critical temperature, the protein underwent denaturation, leading to structural expansion and folding, the temperature at which denaturation occurred was referred to as the denaturation temperature (Tm) or melting temperature. A higher Tm value indicated better thermal stability. The denatured protein structure expanded, exposing hydrophobic regions that bound to the fluorescent dye in solution. Since the fluorescent dyes employed exhibited weak fluorescence in the presence of water but were excited to emit a fluorescent signal in hydrophobic environments, changes in fluorescence intensity of the fluorescent dyes reflected the denaturation of the proteins. The binding of a compound to a protein enhanced the protein's stability, resulting in an elevated Tm value. The Δ_{Tm} value was obtained by subtracting Tm1 (without compound) from Tm2 (with compound); a higher Δ_{Tm} value indicated a stronger binding affinity between the protein and the compound. The method utilized a qPCR instrument to perform melting curve analysis and screen for small molecule ligands. Each target protein possessed a relatively constant melting temperature (Tm) under specific conditions (buffer, pH, ionic strength).

The specific procedure of TSA testing was described as follows: proteins were first dissolved in buffer, and small molecule ligands were dissolved in DMSO. Proteins were mixed separately with small molecule ligands and fluorescent dyes to prepare the experimental samples. Blank and control samples were also prepared, each with 4 replicates. The molar ratio of protein to small molecule ligand was 1:3-1:5. The samples were added to 96-well plates individually, and the temperature was increased from 25° C. to 95° C. at a rate of 1° C./min. Tm and Δ_{Tm} values were determined. The preliminary evaluation results were summarized in Table 4, and the Δ_{Tm} values were presented in Table 4. It is noteworthy that the interaction between all compounds and PAPR14 exhibited a strong positive correlation with protein stability, with Δ_{Tm} greater than 3.

TABLE 4
Δ_{Tm} variation of PARP-14 interaction with target compounds
(TM1-TM6)

| Compound | Inhibition activity against PARP14 (Δ_{Tm}) |
|---|---|
| TM1 | 5.33 |
| TM2 | 5.07 |
| TM3 | 5.05 |
| TM4 | 4.94 |
| TM5 | 4.87 |
| TM6 | 3.48 |

Table 4 listed the test results of the preliminary screening experiments for enzyme inhibition activity of the target compounds. TM1-TM6 were the compounds with the highest inhibition rates, having Δ_{TM} greater than 3, which could be considered to have a strong inhibitory effect. The experimental data suggested that these 6 compounds had a strong interaction with PARP14, indicating that these compounds could act as potent inhibitors of PARP14. Thus, these compounds can inhibit the mono-ADP-ribosyltransferase (MAPR) of target proteins and perform post-genomic regulation of protein activity during cell signaling pathway, which can be further used in the development of precision antitumor drugs.

The structural features and activities of the inhibitors of the target compound PARP14 were analyzed as follows: the most active compound, TM1, with Δ_{TM}=3.25, had methyl and trifluoromethyl or hydroxyl substitutions in its molecular structure, and the two compounds with the second highest inhibitory activity had similar substituents, which also had an electron-withdrawing group of Cl at the meta-position on the aromatic ring attached to the nitrogen atom at the 3-position of the thiazolidinone ring. The spatial distance between 3-Cl or trifluoromethyl substituent on the 3-position benzyl group of 1,3-thiazolidin-4-one and the 4-OH group on the 5-position benzyl of 1,3-thiazolidin-4-one may be critical for inhibiting ADP within the ADP-binding macrodomain of PARP14. The interaction between the substituents that regulate and collectively influence the electron cloud arrangement of the whole molecule likely plays a crucial role in the selective interaction with the target protein. The specific and similar substituent arrangement in the target molecule would be valuable in future design of PAPR14 inhibitors.

### Example 9

### Investigation on Docking Between Target Compounds (TM1-TM6) and Catalytically Active Structures of PARP14 Enzyme

Although more than 20 crystal structures of PARP14 have been published in the Prorein Data Bank (PDB) (Qin, W.; Wu, H.-J.; Cao, L.-Q.; Li, H.-J.; He, C.-X.; Zhao, D.; Xing, L.; Li, P.-Q.; Jin, X.; Cao, H.-L., Research progress on PARP14 as a drug target. Front. Pharmacol. 2019, 10, 172.), considering the presence of a thiazole ring in the structure of the compound described herein, the crystal structure 4PY4, in which the ligand (benzothiazole) contains a similar ring, was selected as the target protein for docking studies (Qin, W.; Li, C.; Wang, R.; Li, P-Q.; Jin, X., Cao, H-L., Virtual screening of PARP14 catalytic domain inhibitors. Clinical Research and Practice. 2020, 5 (30), 3.). First, SYBYL-X 2.0 software was used to build and import a compound library including 6 target compounds, and the compounds were energy optimized with Tripos force field with Gasteiger Hickel charge until the energy no longer decreased. The Surflex-Dock module of the same software was also used to process and optimize the target protein 4PY4, including deletion of the B chain, hydrogenation, and ligand extraction. The active pocket of the PARP14 catalytic domain was selected as the docking site, and a Protomol binding pocket was constructed for docking studies and virtual screening of the compound library. The biological activities of the target compounds were evaluated based on ten scoring parameters, including the total score. Based on the docking results, the molecular mechanism of the best active target compound TM1 interacting with PARP414 was analyzed using Pymol software.

TABLE 1
Results of docking scoring

| | | | | | | | | | | GLOBALC |
|---|---|---|---|---|---|---|---|---|---|---|
| Compound | Total_Score | Crash | Polar | Similarity | Dscore | PMFscore | Gscore | CHEMscore | Cscore | score |
| TM1 | 3.6457 | −3.136 | 0 | 0.262 | −123.732 | −84.9761 | −216.755 | −29.4161 | 4 | 5 |
| TM2 | 3.7607 | −1.0215 | 0.8832 | 0.219 | −105.303 | −55.2066 | −164.509 | −22.3245 | 1 | 2 |
| TM3 | 4.5226 | −1.2468 | 0.2407 | 0.188 | −117.357 | −86.2143 | −204.073 | −31.2271 | 4 | 4 |
| TM4 | 2.7143 | −1.3454 | 1.3068 | 0.281 | −119.299 | −111.749 | −173.712 | −30.1091 | 5 | 3 |
| TM5 | 3.7607 | −1.0215 | 0.8832 | 0.219 | −105.303 | −55.2066 | −164.509 | −22.3245 | 1 | 2 |
| TM6 | 3.4084 | −1.5224 | 1.1048 | 0.258 | −115.086 | −68.9825 | −182.186 | −24.499 | 3 | 4 |

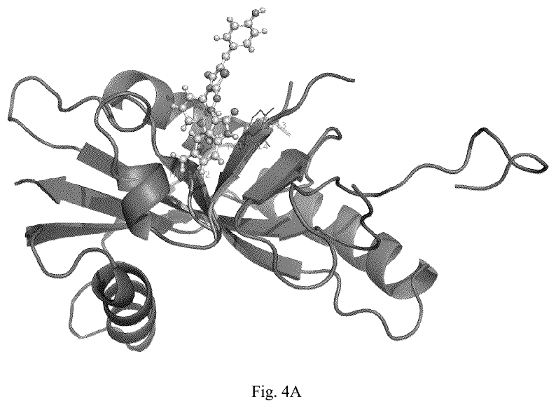

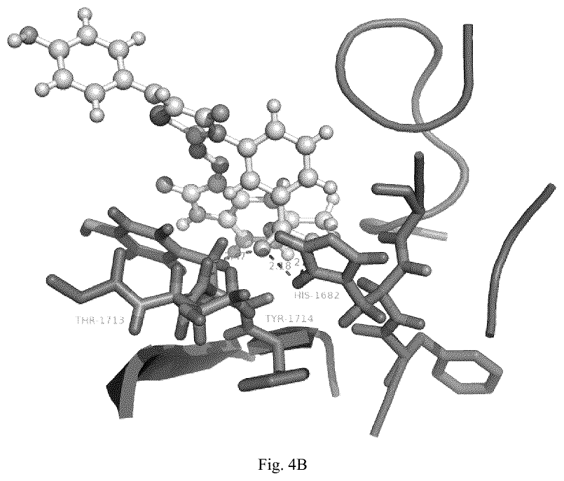

FIG. 4A illustrated an interaction between A-chain holoenzyme of PARP14 and TM1 molecule, and FIG. 4B demonstrated the interaction between the TM1 molecule and a catalytic structural domain of the PARP14.

The docking results indicated that the target compounds were tightly bound to the catalytic domain of PARP14 mainly through hydrogen bonding interactions and aromatic-aromatic interactions. Specifically, there was a π-π stacking interaction (aromatic-aromatic interaction) between an indole ring in the molecular structure of TM1 and an aromatic ring of Tyr1714 in the catalytic domain of PARP14. Moreover, there was a hydrogen bonding interaction between a fluorine atom of 3-CF₃ on the aromatic ring in the molecular structure of TM1 and NH of the amide bond (i.e., peptide bond) backbone between Thr1713 and Tyr1714 (NH . . . F, Distance_{F-H}=2.475 Å), and the fluorine atom also formed a hydrogen bond with the 1-NH of the 1,3-imidazole ring of His1682. Moreover, another fluorine atom of 3-CF₃ also similarly interacted with the 1-NH of the 1,3-imidazole ring of His1682, forming a dual-hydrogen bonding interaction (NH . . . F). Meanwhile, the two hydrogen bonds were of exactly equal bond lengths (2×NH . . . F, Distance_{F-H}=2.311 Å). As a result of the formation of these two hydrogen bonds, a quadrilateral structure constructed from two C—F bonds and two intermolecular hydrogen bonds was formed in the catalytic domain of PARP14, in which the two sides were almost equal in length and the four atoms were almost in one plane when viewed from the side. The first hydrogen bond formed by the amide-peptide bond backbone NH between Thr1713 and Tyr1714 and one of the fluorine atoms (F) was interlinked, and there was also a hydrogen bond (NH . . . F) directly connected to one of the vertices F atoms of this planar quadrilateral, thus constructing a spoon-shaped arrangement similar to the Big Dipper formed by the hydrogen bonds. This mutual characterization between the small molecule and the target provided by the analysis of the molecular docking results may initially confirm the reason for the higher biological activity of the compound at a molecular level. The significant thermal drift of this compound from other molecular structures corresponds to its potential enzyme inhibitory activity, and this difference in activity may be the result of a combination of aromatic interactions and multiple hydrogen bonding interactions between 3-CF₃ on the aromatic ring in the molecular structure of TM1 and amino acid residues Y1714 and His1682 in the catalytic site of PARP14. Meanwhile, these analyses and conclusions mentioned above may also provide a reference for the further design of the structural skeleton and the distribution of substituents of PARP14 small molecule inhibitors.

In conclusion, six target compounds were successfully synthesized by the embodiments of the present disclosure, all of which exhibited good crystallinity and purity, and the chemical structures of all the novel compounds were identified and characterized by ¹H NMR or ¹³C NMR. The inhibitory activity against PARP14 at the enzyme level was tested for the synthesized target compounds. All six target compounds demonstrated strong inhibitory activity, and the Δ_{TM} in the thermal drift assay was greater than 3, which can be employed as lead compounds for the design of PARP14 inhibitors, thus providing theoretical reference and material basis for the design of therapeutic drugs specifically targeting breast tumors.

The above embodiments are merely illustrative of the present disclosure, and are not intended to limit the disclosure. It should be understood that various changes, modifications and replacements made by those skilled in the art without departing from the spirit of the disclosure shall fall within the scope of the disclosure defined by the appended claims.

What is claimed is:

1. An arylidene-indolone-3-substituted thiazolidinone derivative, wherein the arylidene-indolone-3-substituted thiazolidinone derivative is represented by: 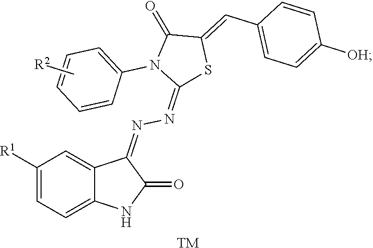
wherein R¹ is a halogen electron-withdrawing group or an alkyl electron-donating group; and
R² is a 3-position or 4-position substituent selected from the group consisting of an electron-withdrawing group and an electron-donating group, wherein the electron-withdrawing group is halogen or trifluoromethyl, and the electron-donating group is tert-butyl.

2. The arylidene-indolone-3-substituted thiazolidinone derivative of claim 1, wherein the arylidene-indolone-3-substituted thiazolidinone derivative is selected from the group consisting of TM1 with R¹ being CH₃ and R² being 3-CF₃, TM2 with R¹ being F and R² being 3-Cl, TM3 with R¹ being CH₃ and R² being 3-Cl, TM4 with R¹ being F and R² being 3-CF₃, TM5 with R¹ being Cl and R² being 4-F, and TM6 with R¹ being Br and R² being 4-C(CH₃)₃.

3. A pharmaceutical composition, comprising:
the arylidene-indolone-3-substituted thiazolidinone derivative of claim 1, or a pharmaceutically-acceptable salt, a hydrate, a solvate, a polycrystal, or a eutectic.

4. The pharmaceutical composition of claim 3, wherein the pharmaceutically-acceptable salt is selected from the group consisting of hydrochloride, hydrobromide, sulfate, phosphate, acetate, methanesulfonate, p-toluenesulfonate, tartrate, citrate, fumarate and malate.

5. A method of preparing the arylidene-indolone-3-substituted thiazolidinone derivative of claim 1, comprising:
(1) reacting 1.0-1.1 equiv. of a substituted aromatic amine (1) with 1.1-1.2 equiv. of chloral hydrate and 3.0-3.3 equiv. of hydroxylamine hydrochloride followed by silica gel column chromatography to collect an intermediate (2);
(2) subjecting the intermediate (2) to cyclization in the presence of concentrated sulphuric acid to obtain 5-substituted isatin (3);
(3) reacting 1.0-1.1 equiv. of a substituted aniline (1.1) with 1.8-2.0 equiv. of CS₂ (4) under an alkaline condition followed by separation and purification to collect a thioglycolate intermediate; subjecting the thioglycolate intermediate to desulfurization in the presence of 1.0-1.1 equiv. of methyl chloroformate and silica gel column chromatography to obtain a substituted aryl isothiocyanate (5);
(4) subjecting the substituted aryl isothiocyanate (5) to hydrazinolysis in the presence of hydrazine hydrate to obtain a N-aryl-substituted thiosemicarbazide intermediate (6);
(5) subjecting 1.0-1.1 equiv. of the 5-substituted isatin (3) and 1.0-1.1 equiv. of the N-aryl-substituted thiosemicarbazide intermediate (6) to intermolecular dehydration condensation in ethanol under the catalysis of concentrated sulphuric acid to obtain a disubstituted thiosemicarbazone intermediate (7); subjecting 1.0-1.1 equiv. of the disubstituted thiosemicarbazone intermediate (7) and 1.0-1.1 equiv. of ethyl 2-chloroacetate to cyclization reaction under the catalysis of anhydrous sodium acetate in an equimolar ratio to obtain an intermediate (8); and
(6) subjecting 1.0-1.1 equiv. of the intermediate (8) and 1.0-1.1 equiv. of p-hydroxybenzaldehyde (9) to a Knoevenagel condensation reaction under the catalysis of anhydrous piperidine, and separation and purification to obtain the arylidene-indolone-3-substituted thiazolidinone derivative, wherein the anhydrous piperidine is 1% of a total number of moles of the intermediate (8) and p-hydroxybenzaldehyde (9);
as shown in the following synthesis route:

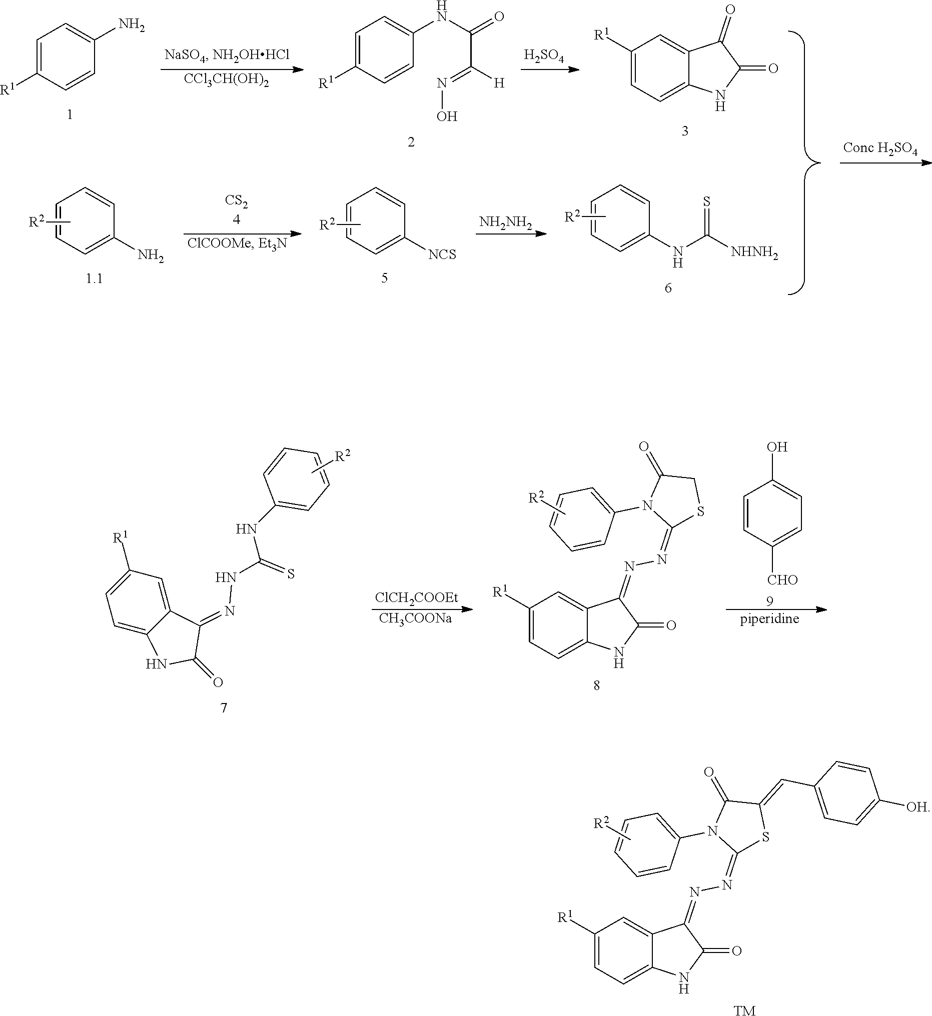

6. The method of claim 5, wherein the arylidene-indolone-3-substituted thiazolidinone derivative is selected from the group consisting of TM1 with R¹ being CH₃ and R² being 3-CF₃, TM2 with R¹ being F and R² being 3-Cl, TM3 with R¹ being CH₃ and R² being 3-Cl, TM4 with R¹ being F and R² being 3-CF₃, TM5 with R¹ being Cl and R² being 4-F, and TM6 with R¹ being Br and R² being 4-C(CH₃)₃.

7. A poly (adenosine diphosphate-ribose) polymerase 14 (PARP14) inhibitor, comprising:
the arylidene-indolone-3-substituted thiazolidinone derivative of claim 1.
